(19) United States
(12) Patent Application Publication
(10) Pub. No.: US 2021/0369900 A1
(43) Pub. Date: Dec. 2, 2021

(54) HYDROGEN PEROXIDE FOAM GENERATING APPARATUS AND RELATED METHOD

(71) Applicant: PeroxiClean Inc., Smithers (CA)

(72) Inventor: Terrance Gordon Fulljames, Smithers (CA)

(21) Appl. No.: 17/325,467

(22) Filed: May 20, 2021

Related U.S. Application Data
(60) Provisional application No. 63/030,360, filed on May 27, 2020.

(51) Int. Cl. A61L 2/22; B01F 3/04; B01F 15/02; B01F 5/06

(52) U.S. Cl. CPC A61L 2/22; B01F 3/04446; A61L 2101/02; B01F 5/0691; B01F 15/0293; B01F 15/0261

(57) ABSTRACT

An apparatus for generating a powerful oxidative hydrogen peroxide foam and chemically stable application process comprises separate supply lines for respectively conveying liquid hydrogen peroxide and liquid foaming agent from separate storage receptacles, a mixer for mixing the liquid hydrogen peroxide and foaming agent once combined together, an air supply line for aerating the foamable liquid hydrogen peroxide mixture, and a foam generating device for mechanically agitating the aerated foamable liquid hydrogen peroxide mixture to form the hydrogen peroxide foam for subsequent discharge to a substrate. There is also disclosed a related method for generating the hydrogen peroxide foam.

This application claims the benefit under 35 U.S.C. 119(e) of U.S. Provisional application Ser. No. 63/030,360 filed May 27, 2020.

### FIELD OF THE INVENTION

The present invention relates to an apparatus and method of generating hydrogen peroxide foam to be subsequently applied to a substrate that is to be chemically treated thereby, for example to remove mold.

### BACKGROUND

It is known to use hydrogen peroxide as an oxidizing agent to remove mold from a substrate surface that is to be treated. Generally the hydrogen peroxide is applied to the surface with the mold thereon in liquid form. The hydrogen peroxide has relatively low concentration, that is generally below 10% concentration. The liquid hydrogen peroxide is left to act on the mold for a prescribed treatment time, during which the mold is substantially killed by chemical action of the hydrogen peroxide. After the prescribed treatment time, the area being treated is mechanically scrubbed to remove residual mold.

### SUMMARY OF THE INVENTION

According to an aspect of the invention there is provided an apparatus for generating hydrogen peroxide foam comprising:

first and second supply lines arranged to respectively convey liquid hydrogen peroxide and liquid foaming agent from separate storage receptacles;

a mixer comprising an intake end arranged in fluidic communication with both the first and second supply lines and arranged to receive the liquid hydrogen peroxide and the liquid foaming agent respectively conveyed by the first and second supply lines, the mixer being configured to blend the liquid hydrogen peroxide and the liquid foaming agent to form a foamable liquid hydrogen peroxide mixture for subsequent release from an outlet end of the mixer in a downstream direction;

an adjustable valve arranged in fluidic communication with the outlet end of the mixer and configured to regulate a rate of flow of the foamable liquid hydrogen peroxide mixture in the downstream direction from the mixer and into an intermediate fluid line;

an air supply line arranged in fluidic communication with the intermediate fluid line downstream from the adjustable valve for introducing air to be mixed with the regulated flow of foamable liquid hydrogen peroxide mixture; and

a foam generating device arranged in fluidic communication with the intermediate fluid line downstream from the adjustable valve and the air supply line, the foam generating device being configured to mechanically agitate the air and the foamable liquid hydrogen peroxide mixture to form the hydrogen peroxide foam for subsequent discharge to a target substrate.

This provides an arrangement capable of mixing constituent ingredients to produce a high concentration hydrogen peroxide foam immediately before discharge to a target substrate, as the high concentration hydrogen peroxide is highly unstable in that it is conducive to decomposition shortly after formation. The hydrogen peroxide foam is an oxidizing agent capable of cleaning a target substrate by chemical action. The foam state of the hydrogen peroxide creates exposure time for the oxidizer to work.

In the illustrated arrangement, each of the first and second supply line includes a restricted orifice arrangement therein configured to limit a flow of a respective one of the liquid hydrogen peroxide and the liquid foaming agent to a prescribed rate to provide a prescribed ratio between the liquid hydrogen peroxide and the liquid foaming agent for blending at the mixer.

In the illustrated arrangement, the restricted orifice arrangement in each of the first and second supply lines is fixed so as to provide a single predetermined flow rate for the respective one of the liquid hydrogen peroxide and the liquid foaming agent.

In the illustrated arrangement, the mixer is a static mixer.

In the illustrated arrangement, the mixer comprises fluidic swivel joints at the intake and outlet ends for operative fluidic connection to the adjustable valve at the outlet end of the mixer and to a coupler at the intake end of the mixer which is in fluidic communication with the first and second supply lines.

In the illustrated arrangement, the mixer comprises a housing defining the intake and outlet ends and a mixing member arranged in the housing for blending of the liquid hydrogen peroxide and the liquid foaming agent in the housing, and both the housing and the mixing member are made from stainless steel.

In the illustrated arrangement, the apparatus includes an adjustable valve along the air supply line configured to regulate a flow of the air to be mixed with the foamable liquid hydrogen peroxide mixture to provide a prescribed ratio therebetween. This airflow regulating device is provided in proximity to the adjustable valve acting as the foamable liquid mixture regulating device so that both of these inputs can be dynamically regulated by an operator, such that the ratio is user-selected.

In the illustrated arrangement, the foam generating device comprises a plurality of interchangeable nozzles having different discharge patterns for discharging the hydrogen peroxide foam to the target substrate.

In the illustrated arrangement, the foam generating device comprises an internal porous body defining a plurality of passageways between an intake end and an output end for permitting passage of the aerated foamable liquid hydrogen peroxide mixture therethrough and an imperforate body covering a portion of the passageways at the output end of the porous body and configured to provide a back pressure at the intake end of the porous body to suitably agitate the air and the foamable liquid hydrogen peroxide mixture upon passage therethrough.

According to another aspect of the invention there is provided a method of applying hydrogen peroxide in the form of a foam to a target substrate comprising:

supplying hydrogen peroxide and foaming agent each in liquid form from a separate storage receptacle;

mixing the supplied hydrogen peroxide and the foaming agent to form a liquid mixture comprising hydrogen peroxide that is foamable;

feeding an airflow to be combined with the foamable liquid mixture;

agitating the airflow and the foamable liquid mixture to form the hydrogen peroxide foam; and

discharging the hydrogen peroxide foam onto the target substrate.

Preferably, a flow of the foamable liquid mixture is dynamically regulated prior to combining the airflow therewith.

Preferably, the airflow is dynamically regulated when feeding to the foamable liquid mixture.

The method preferably further includes selecting one of a plurality of interchangeable nozzles with a prescribed discharge pattern based on the target substrate.

### BRIEF DESCRIPTION OF THE DRAWINGS

The invention will now be described in conjunction with the accompanying drawings in which:

FIG. 1A is a schematic diagram of an upstream portion of an arrangement of apparatus according to the present invention;

FIG. 1B is a schematic diagram of a downstream portion of the apparatus of FIG. 1; and

FIG. 1C is a schematic cross-sectional view along line 1C-1C in FIG. 1B.

In the drawings like characters of reference indicate corresponding parts in the different figures.

### DETAILED DESCRIPTION

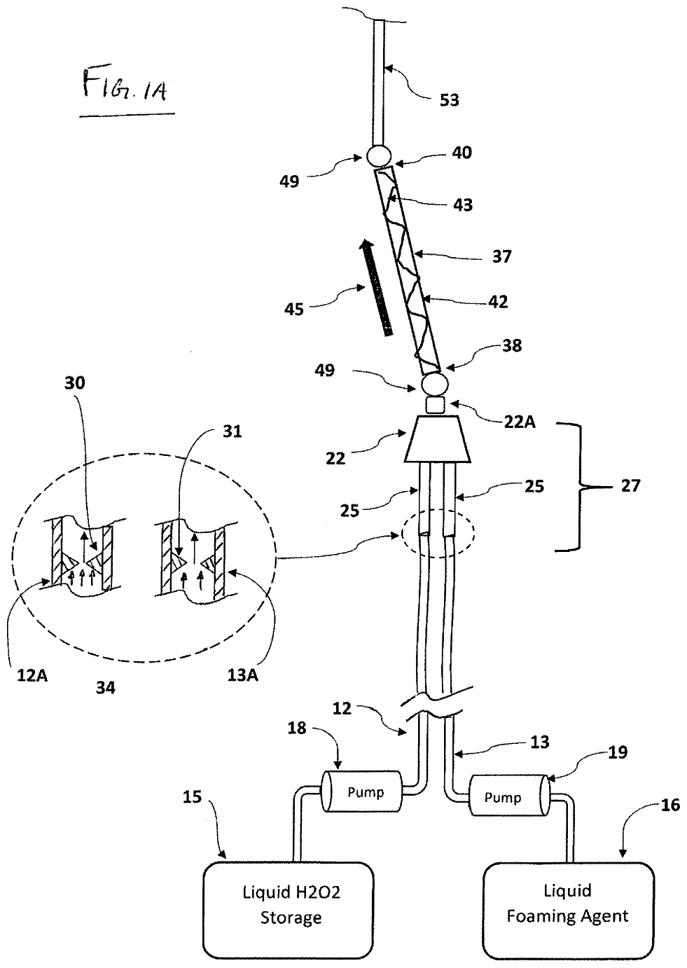

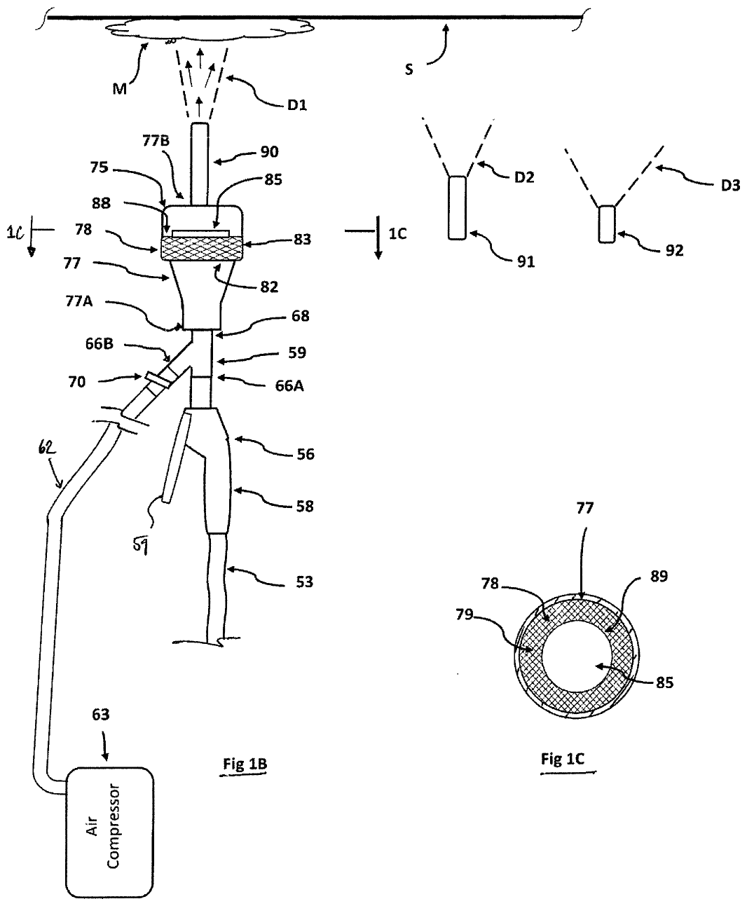

FIGS. 1A and 1B collectively show an apparatus for generating hydrogen peroxide foam for application by spraying to a target substrate S to be treated or cleaned by the hydrogen peroxide in order to remove unwanted living matter carried on the substrate, such as mold M which is a fungus that causes biodegradation of natural materials.

The apparatus comprises first and second supply lines 12 and 13 in the form of hoses arranged to respectively convey liquid hydrogen peroxide and liquid foaming agent from separate storage receptacles 15 and 16 such as tanks. Each storage receptacle 15, 16 which stores or contains only one of the constituent liquids for subsequently forming the hydrogen peroxide foam is in fluidic communication with a distinct pump 18 or 19 which in turn is fluidically communicated with one of the supply lines 12 or 13. Thus the pumps 18, 19 act to move the liquids along the supply lines 12, 13 under pressure. Each pump 18, 19 is set at a different flow rate so as to assist in metering the liquids for later mixing. Additionally or alternatively, an adjustable valve can be disposed along each of the liquid supply lines 12, 13 in series fluidic communication with the respective pump 18 or 19 downstream thereof to assist in regulating a flow of the liquid therealong.

The foaming agent increases a viscosity of the hydrogen peroxide so that when further combined with air the agent will cause the mixture to continuously bubble for a duration of time.

The two supply lines 12 and 13 individually conveying the liquid hydrogen peroxide and foaming agent are merged to a common flow at line coupler 22. Each supply line is fluidically connected to the coupler 22 via an independent check valve 25 which forms with the line coupler 22 a line merging assembly 27 that enables the distinct flows of liquid to combine into the common flow.

For further regulation of the flow rates of each of the constituent liquids for forming the hydrogen peroxide foam, each supply line 12, 13 includes a restricted orifice arrangement 30, 31 in the respective supply lines which is configured to limit a flow of the liquid conveyed thereby to a prescribed flow rate to provide the common flow having a prescribed ratio between the liquid hydrogen peroxide and the liquid foaming agent. In the illustrated arrangement, the restricted orifice arrangement 30, 31 inside each supply line 12, 13 comprises an insert supported by a wall 12A, 13A of the line that defines at least one orifice 33 having an overall or total cross-sectional size which is smaller than that of the supply line defined by the peripheral wall 12A or 13A thereof. Thus the smaller opening 34 provided by the insert acts to reduce the flow of the liquid past the insert to a prescribed rate. Thus the restricted orifice arrangements 30, 31 provide upper limits on the flow rates of the liquid hydrogen peroxide and the liquid foaming agent as set by the pumps 18, 19. The prescribed flow rates of the liquid hydrogen peroxide and the liquid foaming agent are different. Each insert 30, 31 is fixed in shape such that the restricted orifice arrangement defined thereby provides a single predetermined limited flow rate for the respective one of the liquid hydrogen peroxide and the liquid foaming agent.

The common flow end 22A of the line coupler 22 is in fluidic communication with a mixer 37, so that an intake end 38 of the mixer 37 is arranged in fluidic communication with both the first and second supply lines 12, 13 and is thus arranged to receive the constituent liquids respectively conveyed thereby. The mixer 37 is configured to blend or mix together the liquid hydrogen peroxide and the liquid foaming agent to form a foamable liquid hydrogen peroxide mixture for subsequent release from an outlet end 40 of the mixer.

Thus it will be appreciated that, generally speaking, the mixer 37 comprises an intake end 38 configured to receive fluidic material for mixing and an outlet end 40 configured to release mixed fluidic material.

The mixer 37 is a static mixer comprising a housing 42 defining the intake and outlet ends 38, 40, and a mixing member 43 supported in fixed position within the housing 42 which is also stationary and configured to blend or mix the combined flow of constituent liquids as the combined liquid flows through the housing 42 along a flow path indicated by arrow 45 which is defined by the intake and outlet ends 38, 40. In the illustrated arrangement, the housing 42 is tubular and the mixing member 43 is a generally helical flight defining a plurality of mixing vanes which act to mechanically agitate the combined liquid to provide a substantially uniform or homogeneous mixture as the two liquids flow through the mixer housing 42 under the pressure provided by the pumps 18, 19. The mixing member 43 spans substantially a full length of the housing 42 which is about 10 inches and a diameter of an outer edge of the flight spans substantially a full inner diameter of the housing which is about ⅜ inches.

When the hydrogen peroxide and foaming agent are combined they form an exothermic combination which is unstable. Thus, the housing 32 and the mixing member 43 which are effectively the only components of the static mixer 37 are made from stainless steel so as to be suitably resistant to the heat emitted by the combined liquids and to an oxidative power of the high concentration hydrogen peroxide from which the foam is formed, as stainless steel is impervious to hydrogen peroxide. Furthermore, the stainless steel housing 42 is substantially thermally non-conductive such that the heat generated by combination of the liquids is substantially contained within the apparatus.

Since the mixer housing is made of stainless steel such that it is substantially rigid in shape, the mixer 37 includes fluidic swivel joints 49 at the intake and outlet ends 38, 40 for operative fluidic connection to components of the apparatus upstream of the mixer (i.e., the line coupler 22) and downstream thereof relative to the flow of the liquids.

The mixed liquids forming the foamable liquid hydrogen peroxide are released from the outlet end 40 of the mixer and fluidically conveyed by an interconnecting delivery line 53 to a downstream adjustable valve 56 configured to regulate a rate of flow of the foamable liquid hydrogen peroxide in the downstream direction from the mixer 37, that is towards a discharge end of the apparatus, and into an intermediate fluid line 57.

The adjustable valve 56, which is arranged in fluidic communication with the mixer outlet end 40 via the line 53 so as to be fluidically in series with same, is in the form of a fluid control gun comprising a handle 58 arranged for grasping by an operator and a trigger 59 operatively carried on the handle and operatively coupled to a pressure-sensitive spring-tensioned valve inside the gun, which regulates the flow rate in response to pressure applied to the external trigger 59.

An air supply line 62 feeds a pressurized airflow from a pressurized air source 63 such as an air compressor for mixing with the regulated flow of the foamable liquid hydrogen peroxide downstream from the adjustable valve 56. As such, there is provided the intermediate fluid line 57 which forms a manifold in the form of a Y-connector with two intakes 66A and 66B, one of which is fluidically connected to the liquid control gun 56 and the other which is fluidically connected to the air supply line 62, and a single common outlet 68 such that the fluidic flows from the plurality of intakes combine into a common output flow.

The apparatus includes an adjustable valve 70 along the air supply line 62 which is configured to regulate a flow of the air to be mixed with the foamable liquid hydrogen peroxide mixture to provide a prescribed ratio therebetween. This airflow regulating device 70 is provided in proximity to the adjustable valve 56 acting as the foamable liquid mixture regulating device so that both of these inputs can be dynamically regulated by an operator to attain a desired user-selected ratio for desired foaming action.

The single outlet end 68 of the manifold which merges the airflow with the foamable liquid hydrogen peroxide such that the latter is aerated is fluidically coupled with a foam generating device 75, which thereby is in fluidic communication downstream of the adjustable valve 56 and the air supply line 62. The foam generating device 75 is configured to mechanically agitate the air and the foamable liquid hydrogen peroxide mixture to form hydrogen peroxide foam for subsequent discharge to the target substrate S.

In the illustrated arrangement, the foam generating device 75 comprises a housing 77 defining intake and outlet ends 77A, 77B of the foam generating device, an internal porous body 78 in the form of a sponge made from rigid plastic supported in fixed relation in the housing 77 and defining a plurality of passageways 79 between an intake end 82 and an output end 83 of the porous body for permitting passage of the aerated foamable liquid hydrogen peroxide mixture therethrough. The foam generating device 75 further includes an imperforate body 85 in the form of a circular plate covering a portion of the passageways 79, that is some but not all of the passageways, at the output end 83 of the porous body. The imperforate body 85 thus acts as a baffle configured to provide a back pressure at the intake end 82 of the porous body to suitably agitate the air and the foamable liquid hydrogen peroxide mixture upon passage through the porous body. The imperforate body 85 is generally centrally located on the output end 83 of the porous body in spaced relation to the housing 77 so as to define an annular gap 88 between an outer side 89 of the baffle and the housing 77 through which the agitated hydrogen peroxide foam is emitted from the porous body 78 for subsequent discharge from a discharge nozzle 90 of the foam generating device 75.

The foam generating device 75 comprises a plurality of interchangeable nozzles such as those indicated at 90, 91 and 92 having different discharge patterns D₁ through D₃ for discharging the hydrogen peroxide foam to the target substrate S, for example building construction material, to cover a different size and/or shape of surface area. Each nozzle is supportable on the housing 77 in fluidic communication with the outlet end 77B thereof. The various interchangeable nozzles act to control for example hydrogen peroxide foam thickness or distance projection for the discharged hydrogen peroxide foam.

By use of the apparatus of there is provided a method of applying hydrogen peroxide in the form of a foam to a target substrate generally comprising the steps of:

supplying hydrogen peroxide and foaming agent each in liquid form from a separate storage receptacle such as 15, 16;

mixing the supplied hydrogen peroxide and the foaming agent to form a liquid mixture comprising hydrogen peroxide that is foamable, which in the illustrated arrangement is performed at the mixer 37;

feeding an airflow to be combined with the foamable liquid mixture, which is facilitated by air supply line 62 downstream of the mixer 37;

agitating the airflow and the foamable liquid mixture to form the hydrogen peroxide foam, which in the illustrated arrangement occurs at the foam generating device 75; and

discharging the hydrogen peroxide foam onto the target substrate S.

That is, the liquid constituent ingredients for forming the hydrogen peroxide form, namely hydrogen peroxide and foaming agent such as soap, are stored separately from one another without mixing. In this condition the hydrogen peroxide is substantially chemically stable.

The initially separated liquids are subsequently mixed in a prescribed ratio of liquid hydrogen peroxide to liquid foaming agent to obtain suitable foaming action. Each liquid is delivered separately and combined into a common flow, which is then mechanically agitated so that the combined liquid is a substantially uniform mixture.

The foamable liquid, now mixed, is subsequently aerated so that the fluidic mixture has a suitable gaseous component to provide the foaming action. Both the rates of the flow of the foamable liquid mixture and the airflow are dynamically regulated prior to being combined, for suitable foaming action.

After the foamable liquid has been aerated, there is another mechanical agitation step to ensure that the air is mixed well with the foamable liquid before being discharged so that the foam which is sprayed onto the substrate is substantially homogeneous.

Combination of liquid hydrogen peroxide with liquid foaming agent, aeration, and mechanical agitation of the aerated mixture are performed in relatively quick succession and the last step is followed by discharge directly to the target substrate S.

Once the substrate has been treated, no further mechanical action or chemical action is needed to remove residue.

Prior to mixing any of the fluids to form the hydrogen peroxide foam, there may be a step of selecting one of a plurality of interchangeable nozzles 90 through 92 to select a prescribed spray or discharge pattern based on the target substrate to be treated.

Thus is provided an arrangement capable of mixing constituent ingredients to produce a high concentration hydrogen peroxide foam immediately before discharge to a target substrate, as the high concentration hydrogen peroxide is highly unstable in that it is conducive to decomposition shortly after formation. The hydrogen peroxide foam is an oxidizing agent capable of cleaning a target substrate by chemical action. The foam state of the hydrogen peroxide creates exposure time for the oxidizer to work.

As described hereinbefore, the present invention relates to an apparatus wherein two products, namely liquid hydrogen peroxide and liquid foaming agent, are pumped using two independent pumps in separate lines 12, 13 until they merge at a stainless-steel line coupler 22. The two products become one by passing through independent stainless-steel check valves 25 into a solid stainless-steel line coupler 22. This solid stainless-steel line coupler 22 is threaded to mount the check valves 25. Each solution enters the assembly 27 with restricted orifices 30, 31 to mix the products to a specific ratio. The outgoing solution is routed through a threaded hose barb and swivel 49 installed in the top of the solid stainless-steel line coupler 22. The line is connected to a swivel 49 then a static stainless-steel-inline-mixer 37. The static stainless-steel inline-mixer consists of a twisted flat piece of stainless-steel metal defining a mixing member 43 fitted to the inside diameter of a heavy wall stainless steel pipe defining a housing 42 of the mixer. The static stainless-steel-inline-mixer twisted flat bar is welded in place at both ends inside the assembly. After exiting the mixer, the mixed solution enters the line 53 to the fluid control gun 56.

As described, in other words, two products are combined directly after a restricted orifice arrangement beginning in two lines and continuing on in one line. The product is then mixed in a static in-line mixer and moves to the gun valve assembly which regulates flow. Air is added at a predetermined rate by the operator using a valve. The product combined with air is forced through a rigid sponge material primarily plastic and then discharged out the nozzle.

At the mixer, the two products comprising hydrogen peroxide and foaming agent are blended, creating an unstable oxidizer which generates exothermic heat and has a natural bubbling or foaming action even before it reaches the static foam generator or generating device.

The mixed solution enters the fluid control gun. The gun is a pressure-sensitive spring-tensioned valve that opens gradually with applied pressure to the handle. Air is controlled and introduced at the Y connector/valve assembly.

Both air and mixed solution volumes are controlled and constantly modified by the operator. The combined air and mixed solution enter the foam generator and is forced through a plastic foam material. Resistance and back-pressure along with chemical properties of the mixture create controllable hydrogen peroxide foam products and application rates.

The baffle in the foam generator produces back pressure to generate the foam products. The hydrogen peroxide foam is forced out of one of a number of restricting nozzles to match the application.

The hydrogen peroxide foam thus produced has controllable consistency. The foam creates exposure time for the oxidizer defined by the hydrogen peroxide to work, that is to chemically treat or clean the substrate.

Thus the hydrogen peroxide foam essentially consists of hydrogen peroxide, air and foaming agent, none of which are particularly harmful if exposed topically to animals or humans.

The hydrogen peroxide foam does not have any added harmful chemicals and decomposes into oxygen and water such that there is no harmful reside left behind after treatment of the substrate.

Applications of the present invention include:
One step mold-fungal removal and growth inhibitor on all building components;
Powerful cleaner: whole old building shells can be cleaned once interior finishes have been removed;
Sanitizer: for surfaces or areas for example crawlspaces exposed to flood waters; and
Ship hulls/renovations/mold removal for workers; and
Whitens and brightens the appearance of wood, concrete, metal and plastic.

The scope of the claims should not be limited by the preferred embodiments set forth in the examples but should be given the broadest interpretation consistent with the specification as a whole.

1. An apparatus for generating hydrogen peroxide foam comprising:
first and second supply lines arranged to respectively convey liquid hydrogen peroxide and liquid foaming agent from separate storage receptacles;
a mixer comprising an intake end arranged in fluidic communication with both the first and second supply lines and arranged to receive the liquid hydrogen peroxide and the liquid foaming agent respectively conveyed by the first and second supply lines, the mixer being configured to blend the liquid hydrogen peroxide and the liquid foaming agent to form a foamable liquid hydrogen peroxide mixture for subsequent release from an outlet end of the mixer in a downstream direction;
an adjustable valve arranged in fluidic communication with the outlet end of the mixer and configured to regulate a rate of flow of the foamable liquid hydrogen peroxide mixture in the downstream direction from the mixer and into an intermediate fluid line;
an air supply line arranged in fluidic communication with the intermediate fluid line downstream from the adjustable valve for introducing air to be mixed with the regulated flow of foamable liquid hydrogen peroxide mixture; and
a foam generating device arranged in fluidic communication with the intermediate fluid line downstream from the adjustable valve and the air supply line, the foam generating device being configured to mechanically agitate the air and the foamable liquid hydrogen peroxide mixture to form the hydrogen peroxide foam for subsequent discharge to a target substrate.

2. The apparatus of claim 1 wherein each of the first and second supply line includes a restricted orifice arrangement therein configured to limit a flow of a respective one of the liquid hydrogen peroxide and the liquid foaming agent to a prescribed rate to provide a prescribed ratio between the liquid hydrogen peroxide and the liquid foaming agent for blending at the mixer.

3. The apparatus of claim 2 wherein the restricted orifice arrangement in each of the first and second supply lines is fixed so as to provide a single predetermined flow rate for the respective one of the liquid hydrogen peroxide and the liquid foaming agent.

4. The apparatus of claim 1 wherein the mixer is a static mixer.

5. The apparatus of claim 4 wherein the mixer comprises fluidic swivel joints at the intake and outlet ends for operative fluidic connection to the adjustable valve at the outlet end of the mixer and to a coupler at the intake end of the mixer which is in fluidic communication with the first and second supply lines.

6. The apparatus of claim 1 wherein the mixer comprises a housing defining the intake and outlet ends and a mixing member arranged in the housing for blending of the liquid hydrogen peroxide and the liquid foaming agent in the housing, and both the housing and the mixing member are made from stainless steel.

7. The apparatus of claim 1 further including an adjustable valve along the air supply line configured to regulate a flow of the air to be mixed with the foamable liquid hydrogen peroxide mixture to provide a prescribed ratio therebetween.

8. The apparatus of claim 1 wherein the foam generating device comprises a plurality of interchangeable nozzles having different discharge patterns for discharging the hydrogen peroxide foam to the target substrate.

9. The apparatus of claim 1 wherein the foam generating device comprises an internal porous body defining a plurality of passageways between an intake end and an output end for permitting passage of the aerated foamable liquid hydrogen peroxide mixture therethrough and an imperforate body covering a portion of the passageways at the output end of the porous body and configured to provide a back pressure at the intake end of the porous body to suitably agitate the air and the foamable liquid hydrogen peroxide mixture upon passage therethrough.

10. A method of applying hydrogen peroxide in the form of a foam to a target substrate comprising:
supplying hydrogen peroxide and foaming agent each in liquid form from a separate storage receptacle;
mixing the supplied hydrogen peroxide and the foaming agent to form a liquid mixture comprising hydrogen peroxide that is foamable;
feeding an airflow to be combined with the foamable liquid mixture;
agitating the airflow and the foamable liquid mixture to form the hydrogen peroxide foam; and
discharging the hydrogen peroxide foam onto the target substrate.

11. The method of claim 10 wherein a flow of the foamable liquid mixture is dynamically regulated prior to combining the airflow therewith.

12. The method of claim 10 wherein the airflow is dynamically regulated when feeding to the foamable liquid mixture.

13. The method of claim 10 further including selecting one of a plurality of interchangeable nozzles with a prescribed discharge pattern based on the target substrate.
